(19) United States
(12) Patent Application Publication
(10) Pub. No.: US 2024/0199686 A1
(43) Pub. Date: Jun. 20, 2024

(54) C-Terminal Peptide Modification

(71) Applicant: Technische Universiteit Delft, Delft (NL)

(72) Inventors: Rienk Eelkema, Delft (NL); Peggy Bohlaender, Delft (NL)

(21) Appl. No.: 18/281,787

(22) PCT Filed: Mar. 18, 2022
(86) PCT No.: PCT/NL2022/050147

§ 371 Date: Sep. 13, 2023

(30) Foreign Application Priority Data
Mar. 18, 2021 (NL) 2027769

(51) Int. Cl. C07K 1/107; A61K 47/68; C07K 1/10

(52) U.S. Cl. CPC C07K 1/1075; A61K 47/6801; C07K 1/10

(57) ABSTRACT

The invention provides a method for providing a cargo to a C-terminal end of a peptide, the method comprising a first stage and a second stage, wherein the first stage comprises reacting the C-terminal end of the peptide with a first reactant in the presence of a first catalyst and first radiation to provide a first intermediate, wherein the first catalyst is configured to decarboxylate the C-terminal end of the peptide in the presence of the first radiation; and wherein the second stage comprises exposing the first intermediate to a second reactant.

### FIELD OF THE INVENTION

The invention relates to a method for providing a cargo to a C-terminal end of a peptide. The invention further relates to a peptide-cargo conjugate. The invention further relates to a peptide array comprising the peptide-cargo conjugate. The invention further relates to a use of the peptide-cargo conjugate.

### BACKGROUND OF THE INVENTION

Methods for providing a cargo to a protein are known in the art. For instance, US2014256879A1 describes a method for assembling proteins from peptide fragments, wherein a peptide fragment bears a C-terminal cyclic bis(2-sulphanylethyl)amino group.

### SUMMARY OF THE INVENTION

C-terminal modifications of peptides, especially of proteins, may be important for sensors, arrays, (fundamental) research, sequencing applications, as well as for the synthesis of protein-drug conjugates. However, it may be challenging to selectively modify the C-terminal end of native peptides and proteins.

For instance, prior art methods for C-terminal peptide modification may only be compatible with a small range of C-terminal residues.

Prior art methods may further relate to the synthesis of (recombinant) mutant proteins with functional groups for further modifications. However, such methods may not be compatible with modifying peptides from (natural) biological samples.

Further, prior art methods may require solid phase synthesis methods, or may require the use of non-natural coupling chemistry to attach a cargo to the peptide.

Prior art methods may further require specific solvents, which may not be compatible with peptides insoluble in such solvents, which may be particularly relevant with regards to relatively large peptides and proteins.

Hence, prior art methods may be rather restricted with respect to compatibility with peptides, particularly with regards to C-terminal residues, and different types of cargo.

Hence, it is an aspect of the invention to provide an alternative method for the C-terminal peptide modification, which preferably further at least partly obviates one or more of above-described drawbacks. The present invention may have as object to overcome or ameliorate at least one of the disadvantages of the prior art, or to provide a useful alternative.

Hence, in a first aspect the invention may provide a method for providing a cargo to a C-terminal end of a peptide, i.e., a method for providing a peptide-cargo conjugate. The method may comprise a first stage and a second stage. The first stage may especially comprise reacting the C-terminal end of the peptide with a first reactant in the presence of a first catalyst and first radiation to provide a first intermediate. The first catalyst may especially be configured to decarboxylate the C-terminal end of the peptide in the presence of the first radiation. In embodiments, the first reactant may have a first chemical structure according to formula I:

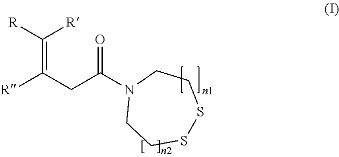

especially wherein R and R′ (or “R′”) are each independently selected from the group consisting of H, and alkyl groups; wherein R″ (or “R″”) is an electron withdrawing group comprising a functional group selected from the group consisting of an ester, a thioester, an amide, a ketone, a nitro, a sulfoxide, a sulfone, a phosphate ester, an acylhydrazide, a cyano group, and a trihalogenmethyl group; and wherein n1 and n2 are each independently selected from the range of 1-2. In embodiments, the second stage may comprise exposing the first intermediate to a second reactant. In embodiments, the second reactant may have a second chemical structure according to formula II:

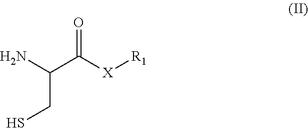

especially wherein X is O or NH, and wherein R1 comprises the cargo. Thereby, the method, especially the second stage, may provide a protein-cargo conjugate.

The method of the invention relates to selectively decarboxylating the C-terminal end of a peptide, which may introduce a radical, and subsequently attaching a reactive group to the C-terminal end of the peptide, especially a bis(2-sulfanylacyl)amido functional group, such as a bis(2-sulfanylethyl)amido functional group. The reactive group may then be used in a second stage to attach a cargo to the peptide. In particular, the method of the invention may provide the benefit that a large variety of cargos, including other peptides, can be selectively covalently linked to the C-terminal end of a natural peptide. This may, for instance, be beneficial in the context of peptide sequencing, especially protein sequencing, in forming antibody drug conjugates, and for providing peptide arrays, especially protein arrays.

In particular, the method, especially the first reactant, may be compatible with most peptides, as C-terminal modification may be possible for peptides having any of the following canonical residues as C-terminal residue: alanine, arginine, asparagine, aspartate, cysteine, glutamate, glutamine, histidine, isoleucine, leucine, lysine, methionine, phenylalanine, proline, serine, threonine, tryptophan, tyrosine, and valine. Further, the method appears also compatible with the non-canonical naturally occurring amino acids pyrrolysine and selenocysteine, as well as with the vast majority of non-natural amino acids.

Further, the method may be executed in an organic solvent, such as DMSO or DMF, as well as in water, which may allow for a larger substrate scope than prior art methods in view of peptide (or cargo) solubilities.

Hence, in specific embodiments, the invention may provide a method for providing a cargo to a C-terminal end of a peptide, the method comprising a first stage and a second stage, wherein the first stage comprises reacting the C-terminal end of the peptide with a first reactant in the presence of a first catalyst and first radiation to provide a first intermediate, wherein the first catalyst is configured to decarboxylate the C-terminal end of the peptide in the presence of the first radiation, and wherein the first reactant has a first chemical structure according to formula I:

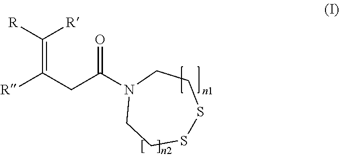

wherein R and R′ are each independently selected from the group consisting of H, and alkyl groups; wherein R″ is an electron withdrawing group comprising a functional group selected from the group consisting of an ester, a thioester, an amide, a ketone, a nitro, a sulfoxide, a sulfone, a phosphate ester, an acylhydrazide, a cyano group, and a trihalogenmethyl group; wherein n1 and n2 are each independently selected from the range of 1-2; and wherein the second stage comprises exposing the first intermediate to a second reactant, wherein the second reactant has a second chemical structure according to formula II:

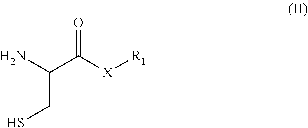

wherein X is O or NH, and wherein R1 comprises the cargo.

Hence, the invention may provide a method for providing a cargo to a C-terminal end of a peptide.

The term “cargo” may herein refer to any compound that may be relevant to attach to a peptide. For instance, the cargo may comprise a second peptide, an antibody, a drug, a transcription factor, a purification tag, a nanoparticle, a fluorescent dye (or “fluorescent probe”), a reporter molecule, and an anchoring tag.

The term “peptide” may herein refer to a chain of amino acids of any length, especially a chain of at least two amino acids, more especially a chain of ten or more amino acids. The term peptide may thus also refer to an oligopeptide, to a polypeptide, and to a protein. In embodiments, the peptide may comprise a natural peptide, especially a natural protein, i.e., a native peptide (or protein) produced by a naturally occurring organism. The peptide may especially comprise a number of amino acids selected from the range of 2-4000, such as 3-2500, especially 3-1000. In further embodiments, the peptide may comprise at most 500 amino acids, such as at most 100 amino acids, especially at most 50 amino acids, such as at most (about) 40 amino acids. In further embodiments, the peptide may comprise at least 2 amino acids, such as at least 3 amino acids, especially at least 5 amino acids.

The method may especially comprise a first stage and a second stage. The term “stage” and similar terms used herein may refer to a (time) period (also “phase”) of a method. The first stage and the second stage may especially be temporally separated, wherein the second stage is temporally arranged after the first stage.

The first stage may comprise reacting the C-terminal end of the peptide with a first reactant in the presence of a first catalyst and first radiation, especially to provide a first intermediate.

The first catalyst may especially be configured to decarboxylate the C-terminal end of the peptide in the presence of the first radiation. In particular, the first catalyst may comprise one or more of riboflavin catalysts, including riboflavin derivatives such as riboflavin tetrabutyrate and lumiflavin, and an iridium catalyst, such as Ir[dF(CF₃)ppy]₂(dtbbpy)PF₆, especially riboflavin tetrabutyrate, or especially lumiflavin. The first catalyst may especially be a photocatalyst decarboxylating the C-terminal end of a peptide when exposed to first radiation. Hence, the first catalyst may be configured to decarboxylate the C-terminal end of the peptide in the presence of the first radiation. The first radiation may especially comprise a first wavelength in the range of 250-600 nm, especially from the range of 300-600 nm. In further embodiments, the first radiation may comprise a first wavelength in the range of 375-525 nm, such as in the range of 400-525 nm. The first radiation may especially comprise a first wavelength selected such that the first catalyst decarboxylates the C-terminal end of the protein when exposed to the first radiation, i.e., the first wavelength may be suitable for the first catalyst to decarboxylate the C-terminal end of the protein when exposed to the first radiation.

In further embodiments, wherein the first solvent (see below) comprises, especially consists of, an organic solvent, the first catalyst may comprise an iridium catalyst selected from the group comprising (Ir[dF(CF₃)ppy]₂(dtbpy))PF₆ (CAS Number 870987-63-6), [Ir(dtbbpy)(ppy)₂]PF₆ (CAS Number 676525-77-2), [Ir(dFppy)₂(dtbbpy)]PF₆ (CAS Number 1072067-44-7), [Ir(dFCF₃ppy)₂-(5,5′-dCF₃bpy)]PF₆ (CAS Number 1973375-72-2), [Ir(dF(Me)ppy)₂(dtbbpy)]PF₆ (CAS Number 1335047-34-1), [Ir{dFCF₃ppy}₂(bpy)]PF₆ (CAS Number 1092775-62-6), [Ir(p-F(Me)ppy)₂-(4,4′-dtbbpy)]PF₆ (CAS Number 808142-88-3), Ir(dFppy)₃ (CAS Number 387859-70-3), Ir(dFFppy)₂(dtbbpy)PF₆ (CAS Number 2042201-18-1), and Ir[dFFppy]₂-(4,4′-dCF₃bpy)PF₆ (CAS Number 2030437-92-2). In such embodiments, the first environment, especially the first solvent, may especially comprise 0.005-2 mM of the first catalyst, such as 0.01-1 mM.

In further embodiments, wherein the first solvent comprises water, or wherein the first solvent comprises an organic solvent/water mixture, the first catalyst may comprise a riboflavin catalyst, including riboflavin derivatives, selected from the group comprising riboflavin tetrabutyrate (CAS Number 752-56-7), Lumiflavin (CAS Number 1088-56-8), Riboflavin (CAS Number 83-88-5), Riboflavin 5′-monophosphate sodium salt (CAS Number 130-40-5), Lumichrome (CAS Number 1086-80-2), riboflavin N-ethyl carbamate, 4-[2-(7,8-dimethyl-2,4-dioxo-3,4-dihydrobenzo[g]pteridin-10(2H)-yl)ethoxy]-4-oxobutanoic acid, 10-Ethyl-3,7,8-trimethyl-benzo[g]pteridine-2,4(3H,10H)-dione, 7,8-dimethyl-10-{2-[bis(2-hydrochloride, 7,8-diethyl-10-{2-[bis(2-hydroxyethyl)amino]ethyl}isoalloxazine hydrochloride, and 7,8-diethyl-10-(1′-d-hydroxyethyl)amino]ethyl}isoalloxazine ribityl)isoalloxazine. In such embodiments, the first environment, especially the first solvent, may especially comprise 0.005-600 mM of the first catalyst, such as 0.01-300 mM.

In further embodiments, the first catalyst may comprise a mesityl acridinium catalyst. In such embodiments, the first environment, especially the first solvent, may especially comprise 0.01-1 mM of the first catalyst.

The first reactant may, in embodiments, have a first chemical structure according to formula I:

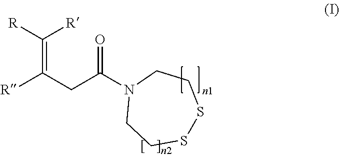

In embodiments, R and R′ may each be independently selected from the group consisting of H, and alkyl groups, especially selected from the group consisting of H and alkyl groups comprising 1-10 C atoms, especially 1-6 C atoms, such as 1-4 C atoms. In particular, the alkyl groups may affect the solubility of the first reactant in different solvents, such as larger alkyl groups reducing the solubility of the first reactant in water. Further, the first reactant may be more reactive (with respect to forming the first intermediate) when R and R′ comprise H or a small alkyl group. Hence, in embodiments, R and R′ may especially be selected from the group consisting of H and alkyl groups comprising 1-6 C atoms, such as from the group consisting of H and alkyl groups comprising 1-4 C atoms, especially from the group consisting of H and alkyl groups comprising 1-2 C atoms. In further embodiments, at least one of R and R′ may comprise H, especially wherein R and R′ both comprise H.

In embodiments, R″ may be an electron withdrawing group, especially an electron withdrawing group comprising a functional group selected from the group consisting of an ester, a thioester, an amide, a ketone, a nitro, a sulfoxide, a sulfone, a phosphate ester, an acylhydrazide, a cyano group, and a trihalogenmethyl group, especially a functional group selected from the group consisting of an ester, sulfoxide, a sulfone, a cyano group, and a trihalogenmethyl group. In particular, the electron withdrawing group may be selected such that the depicted C═C bond in formula I may be (more) reactive with respect to the decarboxylated C-terminal end of the peptide. In further embodiments, the electron withdrawing group may especially comprise an ester group. In further embodiments, the electron withdrawing group may especially comprise a ketone functional group. In further embodiments, the electron withdrawing group may especially comprise a nitro functional group. In further embodiments, the electron withdrawing group may especially comprise a sulfoxide functional group. In further embodiments, the electron withdrawing group may especially comprise a phosphate ester functional group. In further embodiments, the electron withdrawing group may especially comprise an acylhydrazide functional group. In further embodiments, the electron withdrawing group may especially comprise a cyano functional group. In further embodiments, the electron withdrawing group may especially comprise a trihalogenmethyl functional group, especially wherein the trihalogenmethyl functional group comprises halogens selected from the group comprising F, Cl and Br, more especially wherein the trihalogenmethyl functional group comprises three identical halogens, such as a trihalogenmethyl functional group comprising —CF₃, —CCl₃ or CBr₃, especially —CF₃.

In further embodiments R″ may comprise a functional group selected from the group consisting an ester, a cyano group, and —CF₃. In embodiments wherein R″ comprises an ester, R″ may especially comprise a methylester. Hence, in specific embodiments, R″ may comprise a methylester.

In embodiments, n1 and n2 may each be independently selected from the range of 1-2. In particular, during the second stage (see below) the ring (the bis(2-sulfanylacyl)amido functional group) may be opened due to protonation of the sulfur atoms, and the number of carbon atoms between the nitrogen and the sulfur in each “arm” may determine a distance for further reactions, wherein the presence of two carbon atoms between the N and S atoms seems particularly effective. Hence, in further embodiments, at least one of n1 and n2 may be 1, especially wherein both n1 and n2 are 1.

Hence, in specific embodiments, the first reactant may have a first chemical structure according to formula III:

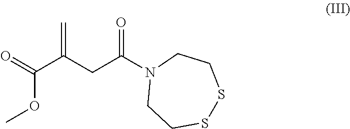 [SMILES: C=C(CC(=O)N1CCSSCC1)C(=O)OC]

A first reactant according to formula III might be particularly beneficial with regards to the method of the invention in view of reactivity and solubility.

In further embodiments, at least one of n1 and n2 may be 2, especially wherein both n1 and n2 are 2.

During the first stage, the peptide may, in embodiments be exposed to a first environment. In particular, the peptide (and the first catalyst) may be arranged in a first mixture comprising a first solvent. In embodiments, the first solvent may especially be selected from the group comprising water, dimethyl sulfoxide (DMSO) and dimethylformamide (DMF), acetonitrile, dimethylacetamide, N-methylpyrrolidone, and tetrahydrofuran, especially from the group comprising water, DMSO and DMF. In further embodiments, the first solvent may (further) comprise an alcohol, especially 1-butanol.

The first mixture may especially comprise one or more of the first solvent, the peptide, and the first reactant. In embodiments, the first mixture may comprise further additives (see below).

In embodiments, the first solvent may comprise water. In such embodiments, the first solvent may have a pH selected from the range of 0.5-8, such as from the range of 0.5-6, especially from the range of 0.5-4.5. In further embodiments, the first solvent may have a pH selected from the range of 1-4, such as from the range of 2-3. Hence, in embodiments, the first environment may have a pH selected from the range of 0.5-8, such as from the range of 0.5-6, especially from the range of 0.5-4.5. In further embodiments, the first environment may have a pH selected from the range of 0.5-4.5, especially from the range of 1-4, such as from the range of 2-3. In particular, it may be beneficial for the first stage to occur in a first environment with a pH of about 2-3, especially of (about) 2.5. In embodiments, the first solvent may comprise at least 70 vol. % water, such as at least 80 vol. %, especially at least 90 vol. %, including 100 vol. %.

In further embodiments, the first environment, especially the first solvent, may have a pH of at least 0.5, especially at least 1. In further embodiments, the first environment, especially the first solvent, may have a pH of at least 1.5, especially at least 2. In further embodiments, the first environment, especially the first solvent, may have a pH of at most 8.5, such as at most 8, especially at most 6, such as at most 4.5. In further embodiments, the first environment, especially the first solvent, may have a pH of at most 4, especially at most 3.5, such as at most 3.

In further embodiments, the first solvent may comprise an organic solvent, especially an organic solvent selected from the group comprising DMSO, acetonitrile and DMF. In embodiments, the first solvent may comprise at least 70 vol. % of the organic solvent, such as at least 80 vol. %, especially at least 90 vol. %, including 100 vol. %. The term “organic solvent” may herein also refer to a plurality of (different) organic solvents.

The first environment, especially the first mixture, more especially the first solvent, may, in embodiments, have a first temperature selected from the range of 5-45° C., especially from the range of 10-40° C. In further embodiments, the method may comprise controlling the first temperature, especially during the first stage, to be at most 45° C., such as at most 40° C. In further embodiments, the method may comprise controlling the first temperature using a temperate control element, especially a cooling element.

In embodiments wherein the first solvent comprises water, the first mixture may further comprise a sequestering agent for reactive oxygen species. In particular, in embodiments, the first mixture may comprise 1-10 vol. % glycerol, such as 3-7 vol. %.

Further, in embodiments, the first solvent may comprise water and an organic solvent, such as 1-butanol. In particular, the first solvent may comprise at least 50 wt. % water and 2-20 wt. % of the organic solvent, such as 5-15 wt. %. The organic solvent may facilitate dissolving of the first catalyst. In further embodiments, the first solvent may comprise a water phase and an organic phase.

In embodiments, the first environment, especially the first mixture, may comprise 0.02-100 mM of the peptide, especially 0.05-50 mM of the peptide.

In further embodiments, the first environment, especially the first mixture, may comprise 0.05-1000 mM of the first reactant, especially 0.1-500 mM.

In embodiments, the first environment, especially the first mixture, may comprise a salt, especially 0.05-100 mM of a salt, such as 1-50 mM of the salt, especially 2-10 mM of the salt. In embodiments, especially embodiments wherein the first solvent comprises an organic solvent, the salt may be selected from the group comprising phosphates, hydrogen phosphates, dihydrogen phosphates, formates, trifluoroacetates, acetates, fluorides and carbonates, especially from the group comprising K₂HPO₄, cesium formate, sodium trifluoroacetate, CsF, CsOAc, Cs₂CO₃, CsF, and K₂CO₃, more especially from the group comprising K₂HPO₄, cesium formate, and sodium trifluoroacetate.

Hence, the first stage may result in a first intermediate. The first intermediate may especially have a formula according to formula 1B:

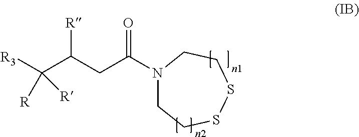

In particular, R₃ may refer to the peptide, and R, R′, R″, n1 and n2 are as defined for the first reactant.

The second stage may comprise exposing the first intermediate to a second reactant. In particular, the second stage may comprise reacting the first intermediate and the second reactant to provide a peptide-cargo conjugate. The second reactant may especially have a second chemical structure according to formula II:

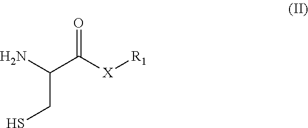

In embodiments, X may be selected from the group comprising O and NH. In further embodiments, R₁ may comprise the cargo.

In particular, the second chemical structure may resemble an N-terminal cysteine residue, optionally bound to the cargo via an amide (CONR), such as in a natural protein with an N-terminal cysteine residue, or optionally bound to the cargo via an ester (COO). In embodiments wherein the cargo is bound via an amide, the R in CONR may refer to H or an alkyl group, especially H or CH₃, more especially H.

In embodiments, during the second stage, the first intermediate may be exposed to a second environment. In particular, the first intermediate (and the second reactant) may be arranged in a second mixture, especially in a second solvent. In embodiments, the second solvent may especially be selected from the group comprising water, dimethyl sulfoxide (DMSO) and dimethylformamide (DMF).

The second mixture may especially comprise one or more of the second solvent, the first intermediate, and the second reactant. In embodiments, the second mixture may comprise further additives (see below).

In embodiments, the second solvent may comprise water. In embodiments, the second solvent may comprise at least 70 vol. % water, such as at least 80 vol. %, especially at least 90 vol. %, including 100 vol. %.

In such embodiments, the second solvent may have a pH selected from the range of 1-11, such as 2-10, especially 2-9. In further embodiments, the second solvent may have a pH selected from the range of 3-7, especially from the range of 3.5-7, such as from the range of 4-6.5. In further embodiments, the second solvent may have a pH selected from the range of 4.5-6.5, especially from the range of 5-6. Hence, in embodiments, the second environment may have a pH selected from the range of 1-11, such as 2-10, especially 2-9. In further embodiments, the second environment may have a pH selected from the range of 3-7, especially from the range of 3.5-7, such as from the range of 4-7. In further embodiments, the second environment may have a pH selected from the range of 4.5-6.5, especially from the range of 5-6. In particular, it may be beneficial for the second stage to occur in an environment with a mildly acidic pH, such as a pH in the range of 3.5-6, especially in the range of 4-5.5. In particular, mild acidic conditions may facilitate an N—S shift that may result in the first intermediate forming a thioester group in situ. The thioester group may especially react with the second reactant to provide the peptide-cargo conjugate.

In further embodiments, the second environment, especially the second solvent, may have a pH of at least 1, especially at least 2, such as at least 3. In further embodiments, the second environment, especially the second solvent, may have a pH of at least 3.5, especially at least 4. In further embodiments, the second environment, especially the second solvent, may have a pH of at most 11, especially at most 10, such as at most 7.5. In further embodiments, the second environment, especially the second solvent, may have a pH of at most 7, especially at most 6.5 such as at most 6, such as at most 5.5.

Hence, in embodiments, during the second stage the first intermediate may undergo an N—S shift providing a thioester group, wherein the thioester group reacts with the second reactant to form the peptide-cargo conjugate.

In particular, during the second stage the first intermediate may be exposed to a reducing agent, especially a reducing agent selected from the group comprising Tris(2-carboxyethyl)phosphine hydrochloride (TCEP), Tris(hydroxymethyl)phosphine (CAS Number 2767-80-8), Tris(hydroxypropyl)phosphine (CAS Number 4706-17-6), Tris(4-methoxyphenyl)phosphine (CAS Number 855-38-9), (4-Hydroxyphenyl)diphenylphosphine (CAS Number 5068-21-3), (2-Hydroxyphenyl)diphenylphosphine (CAS Number 60254-10-6), triphenylphosphine-3,3′,3″-trisulfonic acid trisodium salt hydrate (CAS 335421-90-4), triphenylphosphine-3,3′,3″-trisulfonic acid (CAS 91171-35-6), tris(4,6-dimethyl-3-sulfonatophenyl)phosphine trisodium salt hydrate (CAS 1166403-40-2), trimethyl 3,3′,3″-phosphinetriyltripropanoate (CAS 29269-17-8), tris(3-chlorophenyl)phosphine (CAS Number 29949-85-7), especially TCEP. The reducing agent may reduce the disulfide bond in the first intermediate to provide SH groups, thereby facilitating the N—S-shift. In further embodiments, the second mixture may comprise 10-100 mM of the reducing agent, especially TCEP.

In embodiments, the second mixture may comprise a thiol catalyst, especially an alkane thiol catalyst, or especially an aromatic thiol catalyst. The thiol catalyst may facilitate speeding up the second stage. In further embodiments, the thiol catalyst may comprise one or more of 4-mercaptophenylacetic acid (MPAA; CAS Number 39161-84-7), sodium 2-mercaptomethanesulfonate (MESNA; CAS Number 19767-45-4), and 2,2,2-Trifluoroethanethiol (TFET; CAS Number 1544-53-2), especially MPAA, or especially one or more of MESNA and TFET. In particular, the presence of an alkane thiol catalyst, such as MESNA and TFET, may facilitate a stable conversion at an acidic pH, such as at a pH of about 4.

The formed thioester group may especially react with the second reactant to form the peptide-cargo conjugate.

The second environment, especially the second mixture, may, in embodiments, have a second temperature selected from the range of 25-70° C., such as from the range of 35-55° C.

In further embodiments, the second solvent may comprise an organic solvent, especially an organic solvent selected from the group comprising dimethyl sulfoxide (DMSO), acetonitrile, and dimethylformamide (DMF). In further embodiments, the second solvent may comprise at least 70 vol. % of the organic solvent, such as at least 80 vol. %, especially at least 90 vol. %, including 100 vol. %.

In embodiments, the second environment, especially the second mixture, may comprise 0.05-200 mM of the first intermediate, especially 0.1-100 mM of the first intermediate.

In further embodiments, the second environment, especially the second mixture, may comprise 0.05-400 mM of the second reactant, especially 0.1-200 mM.

The presence of oxygen may be detrimental to providing the first intermediate and/or to providing the protein-cargo conjugate in the second stage. In particular, the presence of oxygen may be detrimental for the photocatalyzed decarboxylation of the peptide in the first stage. In particular, in the first stage, the presence of oxygen may result in a reduced yield of the first intermediate. Further, oxygen may oxidize the S in the first reactant to S═O, which may occur during irradiation with the first radiation. If both S are oxidized to S═O then the N—S-shift to the thioester may no longer be possible in the second stage. Further, in the second stage, the presence of oxygen can also cause oxidation of the SH groups, which can result in closing of the ring by forming the disulfide structure. Hence, in both stages, the presence of oxygen may hamper the method of the invention.

Hence, in embodiments, the first environment, especially the first mixture, may comprise a degassed buffer. In particular, the first environment, especially the first mixture may comprise ≤10 ppm dissolved oxygen, such as ≤5 ppm dissolved oxygen, especially ≤2 ppm dissolved oxygen. In further embodiments, the first environment, especially the first mixture, may comprise ≤1 ppm dissolved oxygen, such as ≤0.8 ppm dissolved oxygen, especially ≤0.6 ppm dissolved oxygen, such as ≤0.5 ppm dissolved oxygen. In further embodiments, the first mixture may comprise at most 0.4 ppm dissolved oxygen, such as 0.2-0.4 ppm dissolved oxygen.

In embodiments, the method may comprise controlling, especially reducing, the (dissolved) oxygen concentration in the first environment, especially in the first mixture, such as in the first solvent. In particular, the method may comprise sparging (or “purging”) the first mixture with an inert gas, especially with N₂, or especially with Ar.

In further embodiments, the method may comprise executing the first stage in a degassed buffer under inert gas, especially N₂ or Ar, especially with Ar. Argon may be particularly suitable as it is heavier than air.

In further embodiments, the second environment, especially the second mixture, may comprise a degassed buffer. In particular, the second environment, especially the second mixture, may comprise ≤10 ppm dissolved oxygen, such as ≤5 ppm dissolved oxygen, especially ≤2 ppm dissolved oxygen. In further embodiments, the second environment, especially the second mixture, may comprise ≤1 ppm dissolved oxygen, such as ≤0.8 ppm dissolved oxygen, especially ≤0.6 ppm dissolved oxygen. In further embodiments, the second mixture may comprise at most 0.4 ppm dissolved oxygen, such as 0.2-0.4 ppm dissolved oxygen.

In embodiments, the method may comprise controlling, especially reducing, the (dissolved) oxygen concentration in the second environment, especially in the second mixture. In particular, the method may comprise sparging the second mixture with an inert gas, especially with N₂, or especially with Ar.

In further embodiments, the method may comprise executing the second stage in a degassed buffer under inert gas, especially N₂ or Ar, more especially Ar. Argon may be particularly suitable as it is heavier than air.

The first stage and the second stage may especially be performed in (essentially) the same solvent, although the solvent may, for instance, differ in pH or salt concentration, such as have a different pH (in the case of water). For example, both the first stage and the second stage may be executed in water, especially wherein a base is added to the first mixture to provide the second mixture. However, in embodiments, the first stage and the second stage may be executed in different solvents. In such embodiments, the method may further comprise an intermediate stage. The intermediate stage may comprise separating the first intermediate, especially from remainder of the first reactant and/or the peptide and/or the first catalyst, especially from the first catalyst, or especially from (remainder of) the first reactant, or especially from (remainder of) the peptide. The intermediate stage may further comprise separating the first intermediate from the first solvent. In embodiments, the intermediate stage may comprise an extraction, especially an extraction with an organic solvent, such as an extraction with one or more of ethyl acetate, dichloromethane, or an ether, such as with an ether selected from the group comprising diethyl ether, methyl tert-butyl ether, and diisopropyl ether.

The intermediate stage may especially be temporally arranged between the first stage and the second stage.

As indicated above, the method of the invention may be compatible with a large variety of C-terminal residues, including (naturally) proteinogenic amino acids, such as canonical amino acids, as well as natural amino acids not naturally incorporated into proteins, such as ornithine, as well as non-natural amino acids. Hence, in embodiments, the peptide may comprise a C-terminal residue selected from the group comprising alanine, arginine, asparagine, aspartate, cysteine, glutamate, glutamine, histidine, isoleucine, leucine, lysine, methionine, phenylalanine, proline, pyrrolysine, selenocysteine, serine, threonine, tryptophan, tyrosine, ornithine and valine, especially from the group comprising alanine, arginine, asparagine, aspartate, cysteine, glutamate, glutamine, histidine, isoleucine, leucine, lysine, methionine, phenylalanine, proline, serine, threonine, tryptophan, tyrosine, and valine.

For some C-terminal amino acids, such as for cysteine and selenocysteine, it may be beneficial to protect the C-terminal amino acid prior to the first stage. Hence, in embodiments, the method may further comprise a preparation stage, temporally arranged before the first stage, wherein the preparation stage comprises protecting a side chain of a residue of the peptide, especially of a cystine residue, or especially of a selenocysteine residue. Methods for capping such residues are known in the art. For instance, the preparation stage may comprise protecting a side-chain of a residue using one or more of iodoacetamide, acrylamide, maleimide, alpha-halo acetamide, and capping by disulfide formation with a small molecule activated disulfide. In particular, the preparation stage may comprise protecting each cysteine and selenocysteine residue in the peptide.

In further embodiments, the preparation stage may comprise blocking a residue, especially a cysteine or selenocysteine residue, using iodoacetamide or maleimide.

In further embodiments, the preparation stage may comprise protecting a residue, especially a cysteine or selenocysteine residue, by forming a disulfide with a thiol.

In embodiments, the cargo may comprise a second peptide. Hence, in embodiments, the peptide-cargo conjugate may comprise a peptide-peptide conjugate, especially a fusion peptide. In further embodiments, the second peptide may have an N-terminal end according to formula II, especially an N-terminal cysteine residue. Hence, the method of the invention may facilitate providing fusion peptides (or proteins), especially wherein the peptide, and optionally the second peptide, is naturally produced. Hence, the method of the invention may facilitate providing a non-recombinant fusion peptide (or protein).

In further embodiments, the cargo may comprise an antibody. In particular, as will be known to the skilled person, antibodies may comprise a plurality of polypeptides, and may thus be attached to the peptide similarly to other peptides. Especially, the antibody may be configured to target a (specific) target location in an animal body, especially in a human body, and the peptide may have a (relevant) medical or therapeutic function at the target location. Thereby, the peptide-cargo conjugate may facilitate providing the peptide to a specific site in an animal body, especially in a human body. Hence, the method of the invention may facilitate providing a peptide-antibody conjugate, especially an antibody-drug conjugate.

In embodiments, the cargo may comprise a tag suitable for selectively arranging a (resulting) peptide-cargo conjugate on a target location.

Various specific examples of cargos are described herein. It will be clear to the person skilled in the art, however, that the invention is not limited to such examples, but may further cover a large variety of other cargo compounds.

In a further aspect, the invention may provide a peptide-cargo conjugate, especially obtainable using the method of the invention. The peptide-cargo conjugate may especially have a chemical structure according to formula IV:

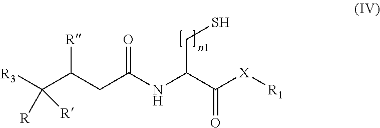

wherein R, R′, R₃, R″, n1, and R₁ may be the same as defined above for the peptide, the first reactant, and the second reactant. In specific embodiments, R₃ may be the peptide; R and R′ may be independently selected from the group consisting of H, and alkyl groups; R″ may be an electron withdrawing group comprising a functional group selected from the group consisting of an ester, a thioester, an amide, a ketone, a nitro, a sulfoxide, a sulfone, a phosphate ester, an acylhydrazide, a cyano group, and a trihalogenmethyl group, n1 may be selected from the range of 1-2, and R₁ may comprise the cargo.

In embodiments, the cargo may comprise an antibody. Hence, the peptide-cargo conjugate may comprise a peptide-antibody conjugate.

In further embodiments, the peptide may comprise (one or more polypeptides of) an antibody. In such embodiments, the cargo may, for instance, comprise a drug, such as a peptide drug. In particular, the C-terminus of an antibody may typically not be involved in guest binding and therefore its functionalization may not interfere with antibody binding. As antibodies generally have 4 C-termini, well-defined cargo loading may be facilitated.

In embodiments, the peptide-cargo conjugate may be for use as an antibody drug conjugate. In particular, the peptide-cargo conjugate may be for use as a medicament, especially wherein the peptide has a medically relevant activity. Antibody-drug conjugates (ADCs) are constructs of an antibody linked with a (cytotoxic) drug, especially via a covalent linker. The purpose of the antibody may be (selective) targeting, and the drug may, for instance, serve to kill tumor (or other) cells. The linker may serve to keep the drug attached to the antibody, such as to avoid releasing the drug in the absence of a target cell or tissue. It may thus be important that the drug is attached via a stable link and in a controllable number on the antibody (which may make attachment to the numerous amine or carboxylate side chains on the antibody less appealing as this may result in heterogenous mixtures and may alter the properties of the antibody). Hence, also considering that the C-terminus of an antibody may typically not be involved in guest binding and therefore its functionalization may not interfere with antibody binding, the method of the invention may be particularly suitable for providing antibody-drug conjugates.

In embodiments, the cargo may comprise a second peptide. Hence, the peptide-cargo conjugate may comprise a fusion peptide, especially a non-recombinant fusion peptide.

In further embodiments, the cargo may comprise a tag suitable for selectively arranging the peptide-cargo conjugate on a target location, such as on a surface. In further embodiments, the tag may comprise an antibody. Such embodiments may facilitate sequencing of the peptide, and may facilitate providing a peptide array

Hence, in a further aspect, the invention may provide a peptide array comprising the peptide-cargo conjugate according to the invention, especially wherein the peptide-cargo conjugate comprises a tag. In particular, peptide arrays may comprise libraries of peptides attached to a solid surface such as a glass slide. Such arrays may be used extensively for the identification of peptide drug candidates, enzyme inhibitors, and enzyme substrates, for profiling antibodies and mapping epitopes and receptor-ligand interactions. The most common method for making these arrays is through covalent attachment, which may require reliable and selective attachment chemistry, such as described in Szymczak et al., “Peptide Arrays: Development and Application”, Analytical Chemistry, 2018, which is hereby incorporated by reference. Relevant chemical groups are usually introduced in peptide synthesis but may be challenging to introduce in native peptides (peptides isolated from biological sources) with prior art methods. The method of the invention, however, may facilitate selective C-term introduction of a suitable chemical group in a peptide to facilitate providing a peptide array. Hence, in embodiments, the cargo may comprise a tag suitable for selectively arranging the peptide-cargo conjugate on a target location, especially on a surface. The tag may, for instance, be suitable for click chemistry. In embodiments, the tag may be selected from the group comprising a polynucleotide (configured to hybridize with a complementary polynucleotide at the target location), a biotin tag (suitable to hybridize with avidin or streptavidin at the target location), and an azide-containing peptide (suitable to be chemically immobilized through click chemistry onto a cyclooctyne-modified surface). It will be clear to the person skilled in the art that many specific choices may be made for the cargo to enable arranging the peptide-cargo conjugate at a target location of a peptide array.

In a further aspect, the invention may provide a use of the peptide-cargo conjugate for peptide sequencing. In particular, next generation peptide/protein sequencing techniques, such as described in Alfaro et al., “The emerging landscape of single-molecule protein sequencing technologies”, Nature Methods, 2021, which is hereby herein incorporated by reference, may involve either immobilization of the peptide on a surface, or linking of the peptide to a molecular unit that will facilitate translocation through a nanopore. For both scenarios, it may be important that a connection is made in a single, specific location on the peptide, to provide reliable orientation on the surface or when passing through the nanopore. As will be clear to the person skilled in the art, a connection to either the N-terminus or the C-terminus of the peptide may be particularly convenient. The method of the invention may facilitate providing a connection between the peptide and the surface or the molecular unit at the C-terminal end of the peptide, thereby facilitating peptide sequencing of the peptide. In particular, in such embodiments the cargo may comprise a polynucleotide, especially a single stranded polynucleotide. The polynucleotide may be configured for arranging the peptide on a surface, such as via hybridization with a (complementary) polynucleotide attached to the surface. The polynucleotide may further be configured to facilitate passing the peptide-cargo conjugate through a nanopore.

The embodiments described herein are not limited to a single aspect of the invention. For example, an embodiment describing the method may, for example, further relate to the (resulting) peptide-cargo conjugate. Similarly, an embodiment of the peptide-cargo conjugate may further relate to embodiments of the method. In particular, an embodiment of the method describing structural formulae with specific groups (e.g., a specific selection of possible R″ groups) may indicate that the peptide-cargo conjugate may, in embodiments, have such groups (such as the specific selection of possible R″ groups).

### BRIEF DESCRIPTION OF THE DRAWINGS

Embodiments of the invention will now be described, by way of example only, with reference to the accompanying schematic drawings in which corresponding reference symbols indicate corresponding parts, and in which:

FIG. 1A-B schematically depict embodiments of the method. The schematic drawings are not necessarily on scale.

### DETAILED DESCRIPTION OF THE EMBODIMENTS

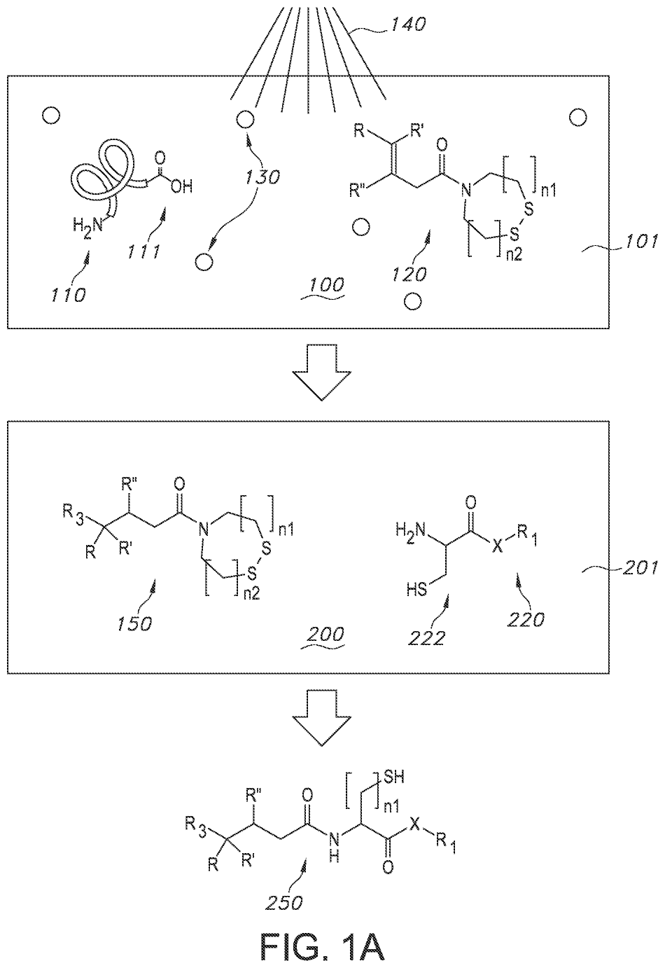

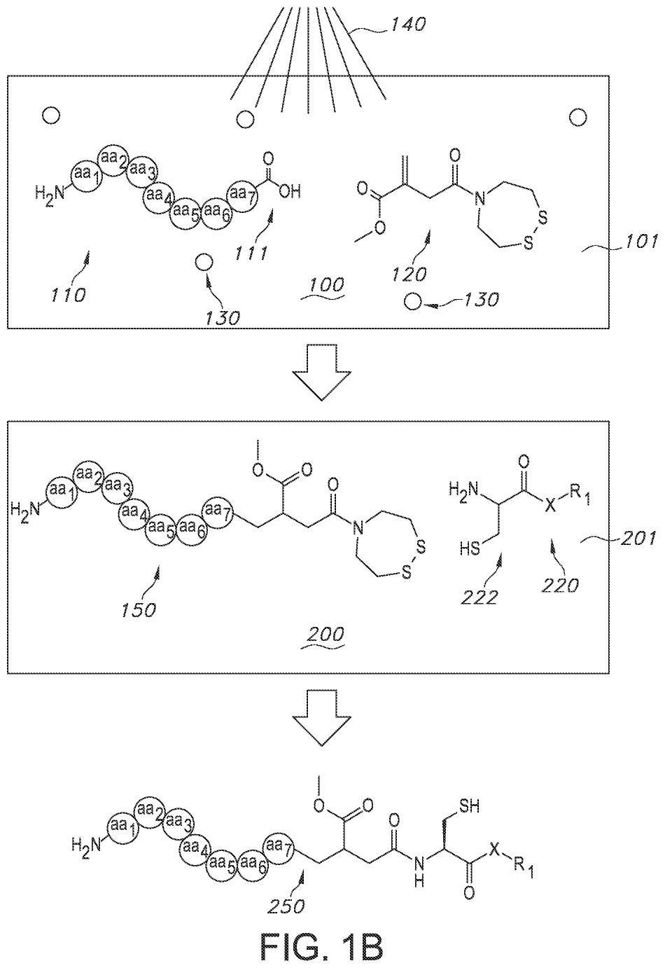

FIG. 1A schematically depicts an embodiment of the method for providing a cargo to a C-terminal end 111 of a peptide 110, i.e., the method for providing a peptide-cargo conjugate. In the depicted embodiment, the method comprises a first stage and a second stage, wherein the first stage comprises reacting the C-terminal end 111 of the peptide 110 with a first reactant 120 in the presence of a first catalyst 130 and first radiation 140 to provide a first intermediate 150. Further, the first catalyst 130 may be configured to decarboxylate the C-terminal end 111 of the peptide 110 in the presence of the first radiation 140. In the depicted embodiment, the first reactant 120 has a first chemical structure according to formula I:

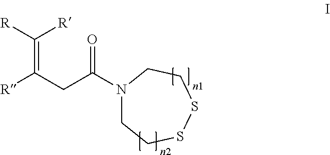

especially wherein R and R′ are each independently selected from the group consisting of H, and alkyl groups; especially wherein R″ is an electron withdrawing group comprising a functional group selected from the group consisting of an ester, a thioester, an amide, a ketone, a nitro, a sulfoxide, a sulfone, a phosphate ester, an acylhydrazide, a cyano group, and a trihalogenmethyl group; and especially wherein n1 and n2 are each independently selected from the range of 1-2.

In the depicted embodiment, the second stage comprises exposing the first intermediate 150 to a second reactant 220, especially wherein during the second stage the first intermediate 150 is exposed to a second environment 200 with a pH selected from the range of 3.5-7. In embodiments, the second reactant 220 may have a second chemical structure according to formula II:

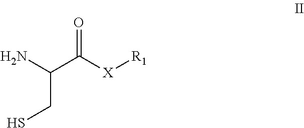

especially wherein X is O or NH, and wherein R1 comprises the cargo. The method of the invention, especially the second stage, may thus comprise reacting the first intermediate 150 with the second reactant 220 to provide a peptide-cargo conjugate.

In embodiments, the first catalyst 130 may be selected from the group comprising riboflavin catalysts, such as riboflavin tetrabutyrate, and iridium catalysts, such as Ir[dFCF₃ppy]₂dtbbpyPF₆, especially wherein the first catalyst comprises riboflavin tetrabutyrate.

The first environment 100 may especially comprise a first mixture comprising a first solvent 101. In embodiments, the first solvent 101 may comprise one or more of DMF, DMSO, and water, especially water. Hence, in embodiments, the first stage may be executed in a first solvent 101.

Similarly, the second environment 200 may especially comprise a second mixture comprising a second solvent 201. In embodiments, the second solvent 201 may comprise one or more of DMF, DMSO, and water, especially water. Hence, in embodiments, the second stage may be executed in a second solvent 201. In further embodiments, the second stage may be performed in a degassed buffer under inert gas, such as under N₂.

In the depicted embodiment, for visualization purposes, only the carboxylic acid group of a C-terminal residue at the C-terminal end 111 of the peptide 110 is depicted. In embodiments, the peptide 110 may comprise a C-terminal residue selected from the group comprising alanine, arginine, asparagine, aspartate, cysteine, glutamate, glutamine, histidine, isoleucine, leucine, lysine, methionine, phenylalanine, proline, pyrrolysine, selenocysteine, serine, threonine, tryptophan, tyrosine, and valine.

In particular, the first stage has been performed using peptides 110 with each of the following C-terminal residues: proline, asparagine, arginine, valine, lysine, leucine, phenylalanine, serine, alanine, and glycine. First intermediates were successfully obtained for each of proline, asparagine, arginine, valine, lysine, leucine, phenylalanine, serine, and alanine.

The second stage has been performed using first intermediates obtained from peptides with asparagine or arginine as C-terminal residues, which in both cases successfully resulted in the peptide-cargo conjugate.

The second stage has further been performed using first intermediates obtained from peptides with proline, valine, lysine, leucine, phenylalanine, serine, and alanine as C-terminal residues, which successfully resulted in peptide-cargo conjugates (see below).

In embodiments, the cargo may comprise a second peptide, especially wherein the second peptide has an N-terminal cysteine residue, i.e., the second reactant 220 may comprise an N-terminal end 222, wherein the second reactant 220 has a cysteine residue at the N-terminal end.

In further embodiments, the cargo may comprise an antibody.

FIG. 1A further schematically depicts a peptide-cargo conjugate obtainable using the method of the invention. In the depicted embodiment, the peptide-cargo conjugate 250 has a chemical structure according to formula IV:

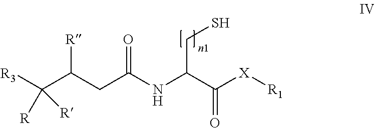

especially wherein R and R′ are each independently selected from the group consisting of H, and alkyl groups; especially wherein R₃ is the peptide, especially wherein R″ is an electron withdrawing group comprising a functional group selected from the group consisting of an ester, a thioester, an amide, a ketone, a nitro, a sulfoxide, a sulfone, a phosphate ester, an acylhydrazide, a cyano group, and a trihalogenmethyl group, especially wherein n1 is selected from the range of 1-2, such as wherein n1 is 1, especially wherein X is O or NH, and especially wherein R₁ comprises the cargo.

In further embodiments, the peptide-cargo conjugate 250 has a chemical structure according to formula IV-2

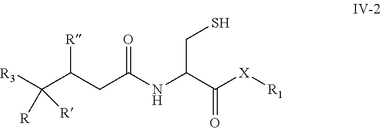

especially wherein R and R′ are each independently selected from the group consisting of H, and alkyl groups; especially wherein R₃ is the peptide, especially wherein R″ is an electron withdrawing group comprising a functional group selected from the group consisting of an ester, a thioester, an amide, a ketone, a nitro, a sulfoxide, a sulfone, a phosphate ester, an acylhydrazide, a cyano group, and a trihalogenmethyl group, especially wherein X is O or NH, and especially wherein R₁ comprises the cargo.

In embodiments, the peptide-cargo conjugate may be for use as a medicament, especially wherein the peptide has a medically relevant (biological) function, and especially wherein the cargo comprises an antibody. In further embodiments, the peptide-cargo conjugate may be for use as an antibody drug conjugate.

FIG. 1B schematically depicts a specific embodiment of the method wherein the first reactant 120 has a first chemical structure according to structure III:

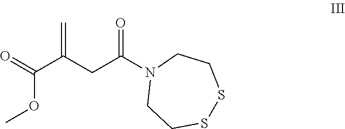 [SMILES: C=C(CC(=O)N1CCSSCC1)C(=O)OC]

In particular, in the depicted embodiment, n1=1, n2=1. Further, in the depicted embodiment, one or more of R and R′ are H. In particular, both R and R′are H. Further, in the depicted embodiment, R″ comprises a functional group selected from the group consisting of an ester, a cyano group, and —CF₃. In particular, R″ comprises an ester; specifically, R″ comprises a methylester.

FIG. 1B further schematically depicts a peptide-cargo conjugate obtainable using the method of the invention. In the depicted embodiment, the peptide-cargo conjugate 250 has a chemical structure according to formula IV:

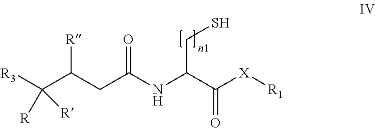

wherein R and R′ are H, wherein R₃ is the peptide, wherein R″ is an ester, especially a methylester, wherein n1 is 1, and wherein R₁ comprises the cargo.

In embodiments, the peptide-cargo conjugate may be for use as a medicament, especially wherein the peptide has a medically relevant (biological) function, and especially wherein the cargo comprises an antibody. In further embodiments, the peptide-cargo conjugate may be for use as an antibody drug conjugate.

### Experiments

The method of the invention was successfully used to provide cargos to C-terminal ends (111) of various peptides (110) using a linker according to structure III:

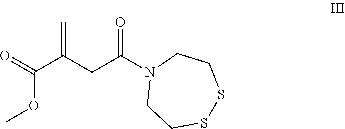 [SMILES: C=C(CC(=O)N1CCSSCC1)C(=O)OC]

Unless specified otherwise, the following procedure was used:
Preparation of peptide stock solution—The peptide was dissolved in anhydrous DMF or DMSO and an amount of peptide stock solution (corresponding to 1 eq, 0.25 μmol) was transferred to a 4 mL dram vial and dried under vacuum (˜1 mbar) at ambient temperature overnight.
Degassing of solvents and solutions—All used solvents (1-butanol, H₂O and DMF) and bigger stock solutions (buffer, glycerol and Riboflavin catalyst in 1-butanol) were degassed separately before being mixed together with the peptide residues in the vials.
First stage—step 1a—Photoreaction—To the peptide solution in the dram vial a magnetic stirring bar, 100 μL glycerol (20% v/v), 25 μL (10.0 eq, 2.5 μmol) 0.1 mol/L cesium formate buffer pH 3.5 and 215 μL water were added. The vial was flushed with argon and dissolving of the peptide was stimulated using an ultra-sonic bath. Afterwards, 20 μL (10.0 eq, 2.5 μmol) of the first reactant (or “linker-molecule”) stock solution (in DMF) was added to the vial and the reaction mixture was degassed for 15 minutes by sparging with nitrogen. Finally, under argon stream, 40 μL (3.0 eq, 0.75μmol) of a degassed Riboflavin catalyst solution (in 1-BuOH) was added and the vial was flushed with argon, parafilmed and the mixture was irradiated for 16 hours using a blue LED (40 W, Kessil, at approximately 4 cm distance).
Addition of thiol—To reduce, especially avoid, an Aza-Michael addition during the drying process a thiol catalyst was added to the mixtures. To the samples 50 eq (12.5 μmol, 0.978 mg, 0.88 μL) 2-Mercaptoethanol was added, and the mixtures were mixed, stirred for ˜30 minutes and then dried under reduced pressure for approximately 90 minutes.
First stage—step 1b—Sample work-up—The residue of step 1a was dissolved in 480 μL H₂O, using the ultrasonic bath, and divided into two parts. Each part was then extracted three times with 800 μL diethyl ether. Then both aqueous layers were dried from diethyl ether under argon stream, were combined, mixed and two portions of 200 μL (max. 0.1 μmol conjugate) were transferred in two separate tubes. (The residual aqueous layer was used for MALDI and LC-MS analysis.) The separated fractions were closed with a perforated cap and freeze-dried.
Second stage—step 2 NCL (Native Chemical Ligation with cargo)—Preparation of the ligation buffer, pH 4.0: 573.2 mg (6 mmol) Guanidine hydrochloride was added to a separate Eppendorf tube and solubilized in 500 μL sodium phosphate buffer (100 mM Na₂HPO₄, pH 9). The whole solution was then transferred to a tube which contained 33.64 mg (200 μmol) 4 Mercaptophenylacetic acid (MPAA) and was dissolved using the ultra-sonic bath. 840 μL of this solution was then added to a tube with 48.16 mg (168.0 μmol) Tris(2 carboxyethyl)phosphine hydrochloride and was mixed until dissolution. To this solution 60 μL 6 M NaOH was added and the solution was mixed. This mixture was adjusted to pH 4.0 using 6 M NaOH. Then the ligation buffer was degassed for 30 minutes by sparging with nitrogen.

Sample preparation—90.0 μL degassed ligation buffer was transferred to the tube which contained the cargo having an N-terminal Cys residue (0.2 μmol cargo as dry residue), and mixed until dissolution. Note: In the following the reaction mixture was flushed with argon any time when the reaction tube was opened. The whole mixture (approximately 6 M guanidine hydrochloride, 200 mM 4-Mercaptophenylacetic acid (MPAA), 200 mM Tris(2-carboxyethyl)phosphine hydrochloride (TCEP), 100 mM sodium phosphate buffer, max. 1.1 mM peptide, 2.2 mM cargo, at pH 4.0.) was then transferred to the tube which contained the material of step 1 and was mixed. Finally, the whole mixture was transferred into a 0.5 mL reaction tube, flushed with argon, closed and packed under argon inside a sealed glass vial and stirred for ˜7-9 days at 50° C.

For HPLC-analysis and purification the reaction mixture was acidified with 45.0 μL 10% TFA in H₂O, mixed and transferred into an 1.5 mL reaction tube. Then the mixture was extracted three times with 1 mL Et₂O in order to remove MPAA. The remaining aqueous layer was injected in the HPLC.

### Experiment 1

Peptide-cargo conjugates were successfully formed with the method of the invention for each combination of peptide in table 1 and cargo in table 2 with the linker according to structure III.

TABLE 1

| Name | Sequence peptide | SEQ ID NO |
|---|---|---|
| MUC5AC | GTTPSPVPTTSTTSAP | SEQ ID NO: 1 |
| TRAP-6 | SFLLRN | SEQ ID NO: 2 |
| ACE I | RPPGFSPFR | SEQ ID NO: 3 |
| MAGE-3 | FLWGPRALV | SEQ ID NO: 4 |
| Mucin 10 | PTTDSTTPAPTTK | SEQ ID NO: 5 |
| OVA | SIINFEKL | SEQ ID NO: 6 |
| Angiotensin III | RVYIHPF | SEQ ID NO: 7 |
| Amylin | SNNFGAILSS | SEQ ID NO: 8 |
| Rhodopsin Epitope Tag | TETSQVAPA | SEQ ID NO: 9 |
| LLO 190 | NEKYAQAYPNVS | SEQ ID NO: 10 |
| IV9, HIV-1 RT Epitope | ILKEPVHGV | SEQ ID NO: 11 |

TABLE 2

| Name | Sequence peptide | SEQ ID NO |
|---|---|---|
| ssrA + cys | CAANDENYALAA | SEQ ID NO: 12 |
| Oxytocin | CYIQNCPLG-NH2 | SEQ ID NO: 13 |

### Experiment 2

Peptide-cargo conjugates were successfully formed with the method of the invention for each combination of peptide in table 3 and cargo in table 4 with the linker according to structure III.

TABLE 3

| Name | Sequence peptide | SEQ ID NO |
|---|---|---|
| TRAP-6 | SFLLRN | SEQ ID NO: 2 |
| Rhodopsin Epitope | TETSQVAPA | SEQ ID NO: 9 |
| Tag | | |

TABLE 4

| Name | Sequence DNA | SEQ ID NO |
|---|---|---|
| DNA-Cargo-1 | 5′-tta ctg aag tct-3′ | SEQ ID NO: 14 |
| DNA-Cargo-2 | 5′-tta ctg aag tct cac | SEQ ID NO: 15 |
| | gtg cct ggt ata tta | |
| | cat cca cag tga att-3′ | |

Both DNA-Cargo sequences were ordered from Biomers.net. The DNA-cargos were provided with a 5′-end cysteine group with a linker according to formula (V):

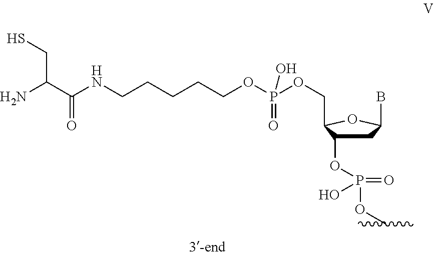

Prior to use, the cysteine groups of the DNA-Cargo sequences was protected with a tertbutylsulfide group, which forms a disulfide with the S of the cysteine group. The addition of TCEP in the second stage results in the reduction of the disulfide bond. Hence, for experiment 2, both DNA-cargos were provided with a 5′-end cysteine.

The term “plurality” refers to two or more. Furthermore, the terms “a plurality of” and “a number of” may be used interchangeably.

The terms “substantially” or “essentially” herein, and similar terms, will be understood by the person skilled in the art. The terms “substantially” or “essentially” may also include embodiments with “entirely”, “completely”, “all”, etc. Hence, in embodiments the adjective substantially or essentially may also be removed. Where applicable, the term “substantially” or the term “essentially” may also relate to 90% or higher, such as 95% or higher, especially 99% or higher, even more especially 99.5% or higher, including 100%. Moreover, the terms “about” and “approximately” may also relate to 90% or higher, such as 95% or higher, especially 99% or higher, even more especially 99.5% or higher, including 100%. For numerical values it is to be understood that the terms “substantially”, “essentially”, “about”, and “approximately” may also relate to the range of 90%-110%, such as 95%-105%, especially 99%-101% of the values(s) it refers to.

The term “comprise” also includes embodiments wherein the term “comprises” means “consists of”.

The term “and/or” especially relates to one or more of the items mentioned before and after “and/or”. For instance, a phrase “item 1 and/or item 2” and similar phrases may relate to one or more of item 1 and item 2. The term “comprising” may in an embodiment refer to “consisting of” but may in another embodiment also refer to “containing at least the defined species and optionally one or more other species”.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The devices, apparatus, or systems may herein amongst others be described during operation. As will be clear to the person skilled in the art, the invention is not limited to methods of operation, or devices, apparatus, or systems in operation.

The term “further embodiment” and similar terms may refer to an embodiment comprising the features of the previously discussed embodiment, but may also refer to an alternative embodiment.

It should be noted that the above-mentioned embodiments illustrate rather than limit the invention, and that those skilled in the art will be able to design many alternative embodiments without departing from the scope of the appended claims.

In the claims, any reference signs placed between parentheses shall not be construed as limiting the claim.

Use of the verb “to comprise” and its conjugations does not exclude the presence of elements or steps other than those stated in a claim. Unless the context clearly requires otherwise, throughout the description and the claims, the words “comprise”, “comprising”, “include”, “including”, “contain”, “containing” and the like are to be construed in an inclusive sense as opposed to an exclusive or exhaustive sense; that is to say, in the sense of “including, but not limited to”.

The article “a” or “an” preceding an element does not exclude the presence of a plurality of such elements.

The invention may be implemented by means of hardware comprising several distinct elements, and by means of a suitably programmed computer. In a device claim, or an apparatus claim, or a system claim, enumerating several means, several of these means may be embodied by one and the same item of hardware. The mere fact that certain measures are recited in mutually different dependent claims does not indicate that a combination of these measures cannot be used to advantage.

The invention also provides a control system that may control the device, apparatus, or system, or that may execute the herein described method or process. Yet further, the invention also provides a computer program product, when running on a computer which is functionally coupled to or comprised by the device, apparatus, or system, controls one or more controllable elements of such device, apparatus, or system.

The invention further applies to a device, apparatus, or system comprising one or more of the characterizing features described in the description and/or shown in the attached drawings. The invention further pertains to a method or process comprising one or more of the characterizing features described in the description and/or shown in the attached drawings. Moreover, if a method or an embodiment of the method is described being executed in a device, apparatus, or system, it will be understood that the device, apparatus, or system is suitable for or configured for (executing) the method or the embodiment of the method, respectively.

The various aspects discussed in this patent can be combined in order to provide additional advantages. Further, the person skilled in the art will understand that embodiments can be combined, and that also more than two embodiments can be combined. Furthermore, some of the features can form the basis for one or more divisional applications.

1. A method for providing a cargo to a C-terminal end of a peptide, the method comprising a first stage and a second stage, wherein the first stage comprises reacting the C-terminal end of the peptide with a first reactant in the presence of a first catalyst and first radiation to provide a first intermediate, wherein the first catalyst is configured to decarboxylate the C-terminal end of the peptide in the presence of the first radiation, and wherein the first reactant has a first chemical structure according to formula I: 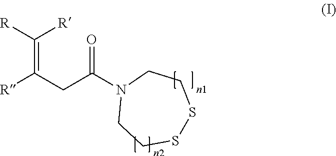
wherein R and R′are each independently selected from the group consisting of H, and alkyl groups;
wherein R″ is an electron withdrawing group comprising a functional group selected from the group consisting of an ester, a thioester, an amide, a ketone, a nitro, a sulfoxide, a sulfone, a phosphate ester, an acylhydrazide, a cyano group, and a trihalogenmethyl group;
wherein n1 and n2 are each independently selected from the range of 1-2;
and wherein the second stage comprises exposing the first intermediate to a second reactant, wherein the second reactant has a second chemical structure according to formula II: 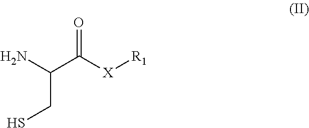
wherein X is O or NH, and wherein R₁ comprises the cargo.

2. The method according to claim 1, wherein n1=1, and wherein n2=1.

3. The method according to claim 1, wherein R and R′ are selected from the group consisting of H and alkyl groups comprising 1-6 C atoms.

4. The method according to claim 3, wherein one or more of R and R′ are H.

5. The method according to claim 1, wherein R″ comprises a functional group selected from the group consisting an ester, a cyano group, or —CF₃.

6. The method according to claim 2, wherein the first reactant has a first chemical structure according to structure III:

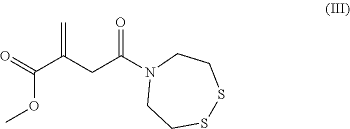 [SMILES: C=C(CC(=O)N1CCSSCC1)C(=O)OC]

7. The method according to claim 1, wherein the first catalyst comprises riboflavin tetrabutyrate.

8. The method according to any one of the preceding claim 1, wherein the first radiation is selected from the range of 375-525 nm.

9. The method according to claim 1, wherein the first stage is executed in a first mixture comprising a first solvent, wherein the first solvent comprises one or more of DMF, DMSO, and water.

10. The method according to claim 9, wherein the first mixture comprises ≤1 ppm dissolved oxygen.

11. The method according to claim 1, wherein the method further comprises an intermediate stage, wherein the intermediate stage comprises separating the first intermediate from the first reactant.

12. The method according to claim 1, wherein the second stage is performed in a degassed buffer under inert gas.

13. The method according to claim 1, wherein the peptide comprises a C-terminal residue selected from the group comprising alanine, arginine, asparagine, aspartate, cysteine, glutamate, glutamine, histidine, isoleucine, leucine, lysine, methionine, phenylalanine, proline, pyrrolysine, selenocysteine, serine, threonine, tryptophan, tyrosine, and valine.

14. The method according to claim 1, wherein the cargo comprises a second peptide, wherein the second peptide has an N-terminal cysteine residue.

15. The method according to claim 1, wherein the cargo comprises an antibody.

16. A peptide-cargo conjugate obtainable using the method of claim 1, wherein the peptide-cargo conjugate has a chemical structure according to formula IV: 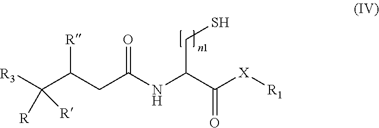
wherein R and R′ are each independently selected from the group consisting of H, and alkyl groups, wherein R₃ is the peptide, wherein R″ is an electron withdrawing group comprising a functional group selected from the group consisting of an ester, a thioester, an amide, a ketone, a nitro, a sulfoxide, a sulfone, a phosphate ester, an acylhydrazide, a cyano group, and a trihalogenmethyl group, wherein n1 is 1, wherein X is O or NH, and wherein R₁ comprises the cargo.

17. The peptide-cargo conjugate according to claim 16 for use as an antibody drug conjugate.

18. A peptide array comprising the peptide-cargo conjugate according to claim 16.

19. Use of the peptide-cargo conjugate according to claim 16 for peptide sequencing.


SEQUENCE LISTING (15 sequences)

SEQ ID NO: 1 - protein, 16 aa, Unknown
  MUC5AC
GTTPSPVPTTSTTSAP

SEQ ID NO: 2 - protein, 6 aa, Unknown
  TRAP-6
SFLLRN

SEQ ID NO: 3 - protein, 9 aa, Unknown
  ACE I
RPPGFSPFR

SEQ ID NO: 4 - protein, 9 aa, Unknown
  MAGE-3
FLWGPRALV

SEQ ID NO: 5 - protein, 13 aa, Unknown
  Mucin 10
PTTDSTTPAPTTK

SEQ ID NO: 6 - protein, 8 aa, Unknown
  OVA
SIINFEKL

SEQ ID NO: 7 - protein, 7 aa, Unknown
  Angiotensin III
RVYIHPF

SEQ ID NO: 8 - protein, 10 aa, Unknown
  Amylin
SNNFGAILSS

SEQ ID NO: 9 - protein, 9 aa, Unknown
  Rhodopsin Epitope Tag
TETSQVAPA

SEQ ID NO: 10 - protein, 12 aa, Unknown
  LLO 190
NEKYAQAYPNVS

SEQ ID NO: 11 - protein, 9 aa, Unknown
  IV9, HIV-1 RT Epitope
ILKEPVHGV

SEQ ID NO: 12 - protein, 12 aa, Unknown
  ssrA+cys
CAANDENYALAA

SEQ ID NO: 13 - protein, 9 aa, Unknown
  Oxytocin
  SITE 9: C-terminal carboxyl group (-COOH) converted to primary amide group (-NH2)
CYIQNCPLG

SEQ ID NO: 14 - DNA, 12 bp, Unknown
  Single-stranded Cys-DNA
  misc_binding 1: /note=“Covalently attached at 5′-end to a linker having a cysteine group”
ttactgaagtct

SEQ ID NO: 15 - DNA, 45 bp, Unknown
  Single-stranded Cys-DNA
  misc_binding 1: /note=“Covalently attached at 5′-end to a linker having a cysteine group”
ttactgaagtctcacgtgcctggtatattacatccacagtgaatt